(19) United States
(12) Patent Application Publication
(10) Pub. No.: US 2021/0077761 A1
(43) Pub. Date: Mar. 18, 2021

(54) LUNG CLEANSING APPARATUS AND METHOD

(71) Applicants: Glenn Fernandes, Thane (IN); Sarita Parikh, Mumbai (IN)

(72) Inventors: Glenn Fernandes, Thane (IN); Sarita Parikh, Mumbai (IN)

(21) Appl. No.: 16/644,303

(22) PCT Filed: Sep. 17, 2018
(86) PCT No.: PCT/IN2018/050600

§ 371 Date: Mar. 4, 2020

(30) Foreign Application Priority Data
Sep. 17, 2017 (IN) 201721032884

(51) Int. Cl. A61M 16/04; A61M 16/00; A61M 16/10; A61H 31/02; A61G 7/018; A61G 7/005

(52) U.S. Cl. CPC A61M 16/0461; A61M 16/0006; A61M 16/1095; A61H 31/02; A61M 2209/10; A61G 7/005; A61M 2210/1039; A61M 2210/065; A61M 2210/0618; A61G 7/018

(57) ABSTRACT

Safe and easy lung cleansing apparatus and method comprising a hollow nasopharyngeal tube with longitudinal misting and air sections inserted in user's nostril, misting means, lung cleansing fluid, fluid and air pumps and automated postural lung cleansing and drainage bed synchronized with the air and fluid pumps, to rotate and turn the user to mist, drain and thereby in turn all areas of the user's lungs. Some embodiments include a cuirass-type negative pressure ventilator to recruit more alveoli for cleansing, loosen mucus in the lungs and induce a cough to clear mucus. The method may include premedication to loosen mucus, tar and other foreign matter in the lungs. In some embodiments, the user's chest could be vibrated to loosen mucus in the lungs to aid thorough cleansing by the misting and automate postural drainage thereafter.

### CROSS-REFERENCE TO RELATED APPLICATIONS

This application claims the benefit of PCT Application No. PCT/IN2018/050600 filed on Sep. 17, 2018.

### FIELD OF INVENTION

This invention relates to the field of lung cleansing; specifically, to an apparatus and method for automated postural lung misting and drainage.

### BACKGROUND OF INVENTION

Chronic lung disease is increasing worldwide, both in developing and developed countries. This is due to increasing active and passive smoking in developed nations and rising levels of air pollution, increasing use of mosquito coils and biomass fuel usage in developing nations. ⅙^{th} of the world smokes and deaths from smoking are the largest preventable deaths in the world. 90% of lung cancers develop because of smoking.

Smoking leaves residues of tar in the lung, which acts as constant mechanical irritants—causing a constant inflammation of lung tissues, and making these lungs more prone to bacterial infection and even cancer. Further, some chemicals in the tar are carcinogens, leading to the development of cancer. If the lungs could be cleaned of this tar residue, it would be of great help in reducing the mechanical and chemical irritation of the lungs, going a long way in improving the lung health of those who smoke. Further, if such a mechanism could be used in a user-friendly, easy manner on a regular basis, it would even allow good health to chronic smokers who are unable to stop smoking. Such a lung cleaning mechanism would also help non-smokers with other types of residues in the lungs, e.g. lung asbestosis.

There are existing lung lavage procedures and prior art, but they require anesthesia and cannot be performed routinely. They also require bronchoscopy and blocking of the bronchi, which can damage the airways, are hospital procedures and can thus even attract superbugs. Further, they wash off the protective lung surfactant and are useful only in rare pulmonary conditions such as pulmonary alveolar proteinosis—a condition characterized by excessive lung surfactant. Further, they use suction to drain the lavaged fluid, which is harmful and can lead to the collapse of the alveoli.

There is thus a need of a lung cleansing procedure that does not harm the smoker's lungs and can be routinely used by those who smoke, or for persons having other types of fine foreign particles in the lungs, or as a routine health procedure that can be performed in a health clinic or even in the user's home.

### SUMMARY OF INVENTION

Embodiments of the present invention are directed to a lung cleansing apparatus and method comprising a hollow nasopharyngeal tube that is inserted into the pharynx via one of the user's nostrils, partitioned to have a first tubular section for injecting a fine mist of a lung cleansing fluid into the lungs and a second tubular section to inject slightly pressurized air into the lungs. The first tubular section comprises a misting means towards its bottom. Also included are a fluid pump connected to the first tubular section for pumping the lung cleansing fluid into the lungs via the first tubular section and an air pump connected to the second tubular section for pumping slightly pressurized air into the lungs; and an automated bed for postural cleansing and drainage that spins, tilts, turns and rotates the user in various postures to facilitate thorough cleaning and drainage of various parts of the lung. The automated postural cleansing and drainage bed movements are synchronized with the fluid pump and the air pump to automatically change the posture of the user based on a preset sequence to enable drainage of one area, followed by change of posture to enable misting of another area and so on till all areas of the lungs have been misted, cleansed and drained.

Other embodiments of the invention include a cuirass type negative pressure ventilator to recruit more lung alveoli for more thorough lung cleansing, to vibrate the chest via high-frequency oscillations in ventilator rate to loosen the mucus and by inducing cough.

In still other embodiments, the user is pre-medicated to loosen the mucus, tar and other foreign particulate matter lodged in the lungs

In some embodiments the user's chest is vibrated by percussion or vibrator belt or other vibration means to loosen the mucus to aid removal of mucus, tar and other foreign particulate matter lodged in the lungs.

The principal object of this invention is to provide an apparatus and method for cleansing the lungs of smoking residues, excess thick secretions, pollutants and other foreign particulate matter lodged in the lungs, without stripping off the lung surfactant or damaging the delicate lung tissue, to reduce the morbidity and mortality due to smoking residues, excess thick secretion buildup and other foreign particulate matter lodged in the lungs and that can be safely and easily performed by the user alone, without mandating the use of anesthesia or skilled medical personnel

Another object of this invention is to provide a suitable apparatus and method to cleanse the lungs without the use of suction to drain the cleansing fluid from the lungs.

Another object of this invention is to provide suitable apparatus and method to loosen the mucus in the lungs to help easier cleansing of the lungs.

Another object of this invention is to provide a suitable apparatus and method to cleanse the lungs without gagging the user or injuring the airways.

Another object of this invention is to provide suitable apparatus and method to cleanse even the normally collapsed alveoli of the lungs.

Another object of this invention is to provide a suitable apparatus and method to automate the process of cleansing and draining different parts of the lung.

Another object of this invention is to provide suitable apparatus and method to allow normal breathing in both lungs during the entire process of cleansing of the lungs.

Another object of this invention is to provide a suitable apparatus and method to prevent damage or discomfort to the lungs during the lung cleansing process by the cooling of the lungs.

Another object of this invention is to provide suitable apparatus and method to allow inexpensive cleansing of the lungs.

Another object of this invention is to provide suitable apparatus and method to stimulate mucociliary clearance in the lungs.

Another object of this invention is to provide suitable apparatus and method to avert the need for hospitalization for the cleansing of the lungs.

While the invention is described herein by way of example using several embodiments and illustrative drawings, those skilled in the art will recognize that the invention is not limited to the embodiments of drawing or drawings described, and are not intended to represent the scale of the various components. Further, some components that may form a part of the invention may not be illustrated in certain figures, for ease of illustration, and such omissions do not limit the embodiments outlined in any way. It should be understood that the drawings and detailed description thereto are not intended to limit the invention to the particular form disclosed, but on the contrary, the invention is to cover all modification, equivalents, and alternatives falling within the spirit and scope of the present invention as defined by the appended claims. The headings used herein are for organizational purposes only and are not meant to be used to limit the scope of the description or the claims. As used throughout this application, the word “may” is used in a permissive sense (i.e., meaning having the potential to), rather than the mandatory sense (i.e., meaning must). Similarly, the words “include,” “including,” and “includes” mean including, but not limited to. Further, the words “a” or “an” mean “at least one” and the word “plurality” means one or more, unless otherwise mentioned. Various objects, features, aspects, and advantages of the present invention will become more apparent from the following detailed description of preferred embodiments of the invention, along with the accompanying drawings in which like numerals represent like components.

### BRIEF DESCRIPTION OF THE DRAWINGS

So that the manner in which the above-recited features of the present invention can be understood in detail, a more particular description of the invention, briefly summarized above, may be had by reference to embodiments, some of which are illustrated in the appended drawings. It is to be noted, however, that the appended drawings illustrate only typical embodiments of this invention and are therefore not to be considered limiting of its scope, for the invention may admit to other equally effective embodiments.

These and other features, benefits, and advantages of the present invention will become apparent by reference to the following text figures, with like reference numbers referring to like structures across the views, wherein:

FIG. 1A illustrates an outline view of the lung misting apparatus and a cross-section of the human nasopharynx and lungs showing how the apparatus will be positioned, according to one embodiment of the present invention;

FIG. 1B illustrates an outline view of the lung misting apparatus according to one embodiment of the present invention;

FIG. 2 illustrates different body postures in which the user can be positioned during lung misting and drainage according to one embodiment of the present invention; and

FIG. 3 illustrates a perspective view of the automated bed for postural cleansing and drainage, illustrating the rotation axes according to one embodiment of the present invention.

### DETAILED DESCRIPTION OF THE INVENTION

The present invention is a novel apparatus and method of cleansing the lungs of fine particulate foreign matter like tar residue, fine particles of asbestos etc. The procedure is simple and can be routinely performed by the user himself, without the need of hospitalization and the costs are thus reduced, without disrupting the user's day-to-day activities. In addition of cleansing the lung of tar and other residues of cigarette smoke/environmental pollutants, such a device if used with lung cleansing fluids that also improve mucociliary clearance like hypertonic saline, for example, would also be useful in users with cystic fibrosis and other conditions leading to excessive secretion buildup in the lung, improving the quality of life and even reducing morbidity and mortality in such persons. Various embodiments of the invention are described below.

The apparatus for lung cleansing disclosed in this invention includes a means to inject a fine mist of desired lung cleansing fluid into the lungs and an automated bed for postural cleansing and drainage, which would help clean all lobes of the lung. It also preferably consists of a means to pump slightly pressurized air into the lungs. It could also include an automated chest percussion system. Other accessories could include chest vibration means, devices to increase inflation of lungs and induce a cough.

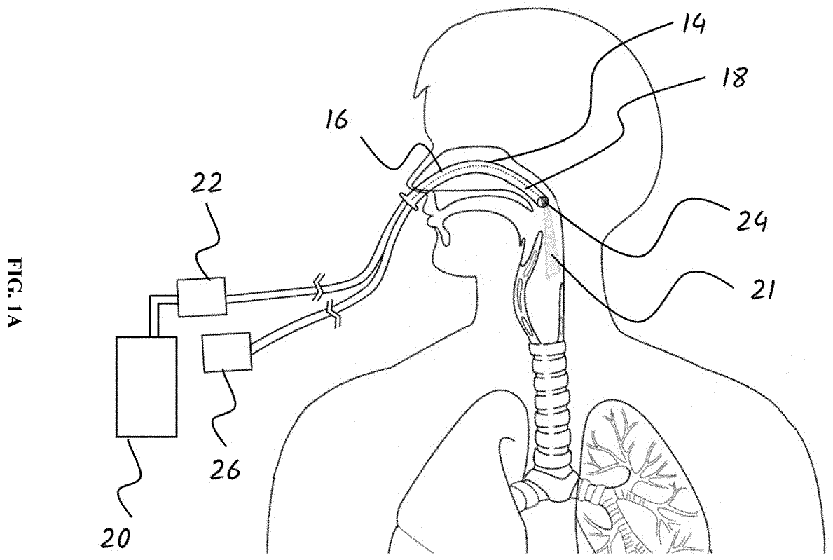

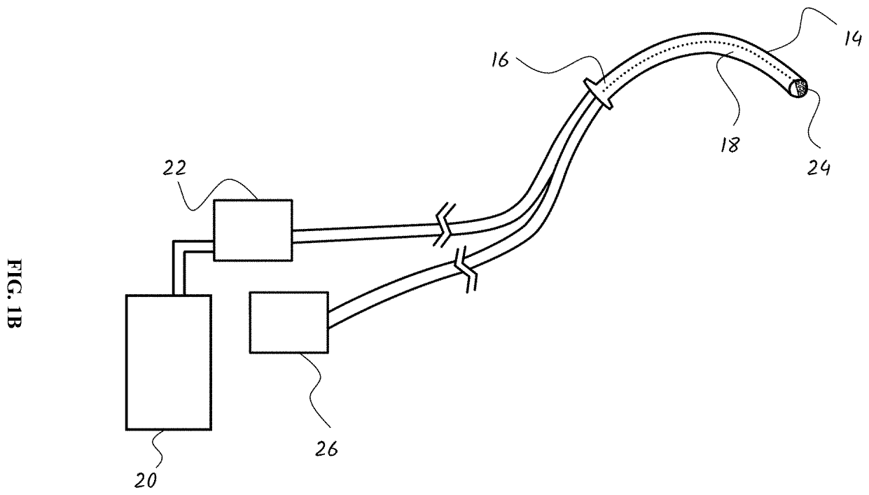

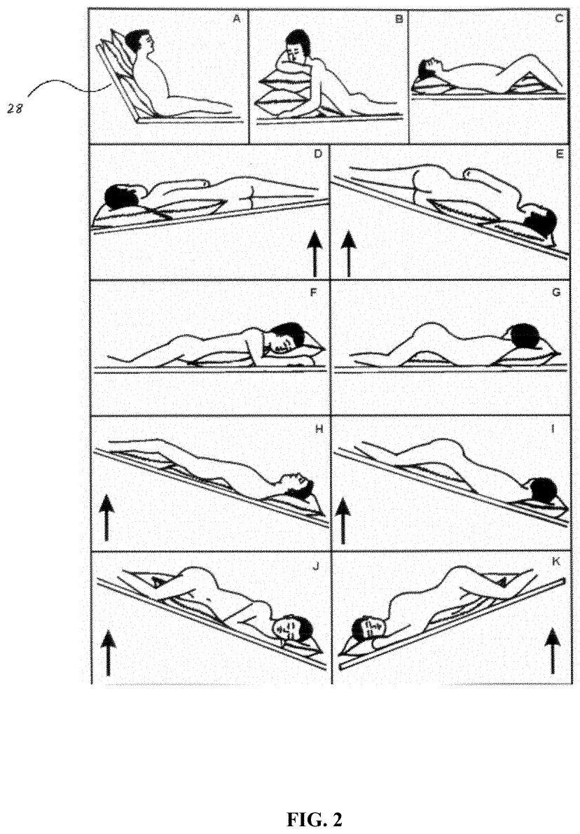

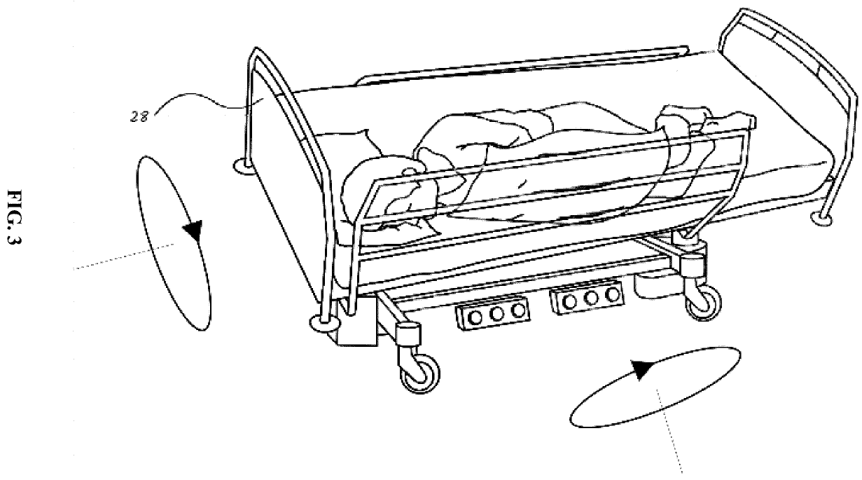

In an embodiment illustrated in FIGS. 1A and 1B, the lung cleansing apparatus includes: a hollow nasopharyngeal tube 14 with a longitudinal partition 15 wherein the nasopharyngeal tube is adapted to be placed into a pharynx via a nostril, wherein the hollow nasopharyngeal tube 14 includes a first tubular section 16 for injecting a lung cleansing fluid 20 which is misted and sprayed as a fine fluid mist 21 into a lung, by means of a mist generator 24 as fine orifice holes preferably towards the bottom of first tubular section 16, a second tubular section 18 adapted to provide passage to injected air into the lung along with fluid mist 21; a fluid pump 22 coupled to the first tubular section 16 for pumping the fluid mist 21 into the lung via the first tubular section 16; an air pump 26 connected to the second tubular section 18 for pumping air into the lung. This lung cleansing fluid 20 could be composed of isotonic saline or hypertonic saline or any other suitable lung cleansing fluid and could include medication to loosen mucus/help cleanse the tar and other foreign residues. Several lung cleansing fluids could also be used sequentially for a thorough cleansing. FIG. 2 and FIG. 3 illustrate an automated bed 28 for postural cleansing and drainage, which includes a plurality of sides that is configured to pivot around any of the plurality of sides to enable tilting of the automated bed 28 into a plurality of positions at different angles with respect to a horizontal axis; and a control unit that signals the automated bed 28 to switch positions in a predetermined sequence on at least one of (a) turning on and off of the fluid pump 22 or the air pump 26 or (b) at least one predetermined interval.

In an embodiment, the lung cleansing apparatus, comprises a cuirass-type negative pressure ventilator adapted to be fixed to at least a part of a thoracic-abdomen and configured to apply a negative pressure in a range of 0 to 50 cm of H₂O and a positive pressure in the range of 0 to 50 cm H2O.

In an embodiment, the lung cleansing apparatus, includes: a heater to preheat at least one of:

(a) the air pumped in via the second tubular section 18; or

(b) the lung cleansing fluid 20 pumped in via the first tubular section 16.

In an embodiment, the lung cleansing apparatus includes at least one joint in the automated bed 28 which divides the automated bed 28 into at least two sections out of which at least one section folds in a range of 5 degrees to 90 degrees through the joint in relation to a horizontal plane of the automated bed 28.

In an embodiment, the lung cleansing apparatus includes an ultrasonic device coupled to an end of the nasopharyngeal tube as mist generator 24, to create a fine fluid mist 21 from the lung cleansing fluid 20.

In an embodiment, the system includes: an automated calibrated bed enabled to change position of an occupant wherein the said automated calibrated bed comprises a plurality of sides, and is configured to pivot around any of the plurality of sides to enable tilting of the automated calibrated bed to a plurality of positions at different angles with respect to a horizontal axis; a pressurized pump device; a control unit for receiving a signal from the pressurized pump device when the pressurized pump device is turned on, wherein the pressurized pump device is coupled with nasopharyngeal tube, wherein the nasopharyngeal tube includes, a first tubular section 16 which is adapted for injecting a fine fluid mist 21 into a lung, wherein the first tubular section 16 includes an opening at one end of the first tubular section 16 for the fluid mist 21 to enter the lung; a pressurized air part 18 which is used to inject air with predetermined pressure into the lung along with the fluid mist 21; a fluid pump 22 coupled to the first tubular section 16 for pumping the lung cleansing fluid 20 into the lung via the first tubular section 16; an air pump 26 connected to the second tubular section 18 for pumping air with predetermined pressure into the lung; changing a position of the automated calibrated bed from a first position around a first axis to a second position around a second axis on an opposite side of the automated calibrated bed on receiving a signal from the control unit.

In an embodiment, the automated position shifting bed control system further includes at least one joint in the automated bed 28 which divides the automated bed 28 into at least two sections out of which at least one section folds in a range of 5 degrees to 90 degrees through the joint in relation to the horizontal plane of the automated bed 28.

In an embodiment, a method for operating an apparatus, includes the steps of: activating, a cuirass-type negative pressure ventilator device that is placed next to at least a part of a thoracic-abdomen with a negative pressure in a range of 0 to 50 cm of H2O and a positive pressure in the range of 0 to 50 cm of H2O; placing, a hollow nasopharyngeal tube 14 with a longitudinal partition 15 into a pharynx via a nostril, wherein the hollow nasopharyngeal tube 14 comprises: a first tubular section 16 which is used for injecting a fluid mist 21 into a lung, wherein the first tubular section 16 includes, an opening at one end for the fluid mist 21 to enter the lungs; a second tubular section 18 which provides a passage to injected air into the lung along with the fluid mist 21; pumping, with a fluid pump 22 connected to the first tubular section 16 the fluid mist 21 into the lungs via the first tubular section 16; pumping, with an air pump 26 connected to the second tubular section 18 air with predetermined pressure in the range of 4 cm of H₂O to 6 cm of H₂O into the lungs; pivoting, an automated bed 28 including a plurality of sides, around any of the plurality of sides to enable tilting of the bed to an angle that ranges from 5 to 80 degrees to horizontal axis; signaling, at a control unit, to switch a position of the automated bed 28 in a predetermined sequence on at least one of (a) turning on and off of the fluid pump 22 or the air pump 26 or (b) on completion of a predetermined interval.

In an embodiment, the method further includes preheating, with a heater at least one of, the air pumped in via the second tubular section 18; or the lung cleansing fluid 20 pumped in via the second tubular section 16.

In an embodiment, the method further includes, changing a position of the automated bed 28, wherein at least one joint in the automated bed 28 divides automated bed 28 into at least two sections out of which at least one section folds in a range of 5 degrees to 90 degrees through the joint in relation to a horizontal plane of the automated bed 28.

Some embodiments could have additional chest vibrating means to loosen the mucous in the lungs. Some embodiments could have vibration belt fixed around the user's chest, other embodiments have a cuirass negative pressure ventilator worn by the user, which could be alternately subject to negative and positive pressure, at a suitable frequency, to cause chest vibration enough to loosen the lungs, or any other suitable chest percussion and vibration means. Vibration could be done both prior to and during the procedure. Additionally, the cuirass negative pressure ventilator worn by the user could be used to increase the recruitment of lung alveoli, as is seen in negative pressure ventilation, to help more thorough cleansing of lung and additionally it could also help to assist coughing out the loosened mucus by raising the positive expiratory pressures in cough mode of the negative pressure ventilation to help user drain this lung misting fluid and mucous when required. Optionally, the user could be pre-medicated with mucolytic agents to help make the secretions less viscous, easy to drain. The user would lie on an automated bed 28 that will change the position of the user into various postures suitable for drainage of fluid containing tar and other fine residue deposited in the lungs from different areas of the lung as illustrated in FIG. 2 and FIG. 3. The automated bed 28 could be timed with the pumps 22 and 26, such that when the pumps are turned off after sufficiently misting one area of the lungs, the automated bed 28 could automatically change the posture of the user based on a preset sequence, to enable drainage of that area, followed by change of posture to enable misting of another area and so on, till all areas of the lungs have been misted, cleansed and drained. All drainage of lung cleansing is only by postural means—there is no suction used to drain the lung cleansing fluid out of the lungs, thus preventing the collapse of the lungs and discomfort to the user that could be caused by suction of lung cleansing fluid. The use of fine fluid mist 21 by embodiments precludes removal of protective lung surfactant and lung damage which could otherwise occur if the lung were washed with the fluid being poured into the lungs instead of it being misted into the lungs. Since a short hollow nasopharyngeal tube 14 is used, which can be self-inserted by the user using only a lubricant, without the need of anesthesia, there is no need for skilled medical personnel for the procedure. There is also no danger of injury to the larynx or airway as the hollow nasopharyngeal tube 14 is very short, stopping in the user's pharynx. The short hollow nasopharyngeal tube 14 being nasopharyngeal, there is no gagging, and no danger of bronchospasm and the fluid can be drained by postural means. Since there is no anesthesia, the user is ready to resume his activities as soon as the procedure is over.

The method for lung cleansing disclosed in the present application includes the following steps:
1. Pre-medicate
   Pre-medicate the user with oral/inhaled mucolytic agents if required.
2. Chest Vibration
   Use suitable means like a vibratory belt, or percussion or cuirass-type negative pressure ventilator to vibrate the chest—preferred to loosen the mucous in the lungs. The vibration can start before the procedure and can continue throughout the procedure.
3. Connect the pumps and insert the nasopharyngeal tube
   The user can self-insert the hollow nasopharyngeal tube 14 via through one nostril after suitable lubrication. The first tubular section 16 is connected to the fluid pump 22 and the second tubular section 18 is connected to air pump 26.
4. Posture of the user
   The user is positioned in a series of different positions throughout the procedure, to cleanse and drain different areas of the lung by means of the automated bed 28, which spins, rotates and turns the patient to allow thorough misting and drainage of all parts of the user's lungs.
5. The misting and drainage procedure: In an embodiment, the fluid pump 22 pumps lung cleansing fluid 20 into the first tubular section 16, at predetermined rate and pressure, which is preferably adjustable even during the procedure. Second tubular section 18 simultaneously pumps slightly pressurized air around 4-6 cm H2O pressure or whatever pressure is comfortable to the user, to maintain a slight positive pressure in the lungs throughout the process, helping keep the airways and alveoli open throughout the process, ensuring adequate ventilation and drainage of the lungs. The fine fluid mist 21 is sprayed into the lungs. In an embodiment, the mist is hypertonic saline or some such fluid that increases mucociliary clearance. The air and/or lung cleansing fluid 20 could be slightly pre-heated if required, before entering the lungs. The air pumped in could have a required amount of oxygen in users with impaired oxygenation. Fine aerosolized lung cleansing fluid 20 is misted as fluid mist 21 into the lungs. The user is kept in one posture for few minutes and then the device is turned off and the user's posture is changed by the automated bed 28 to drain the fluid from that part of the lung. The posture is changed again by the automated bed 28 to mist another area of the lung and the procedure followed above is repeated till all the areas of the lung are misted and drained. The automated bed 28 movements are synchronized with the fluid pump 22 and the air pump 26 to automatically change the posture of the user based on a preset sequence to enable drainage of one area, followed by change of posture to enable misting of another area and so on till all areas of the lungs have been misted cleansed and drained. Throughout this procedure, the chest vibration/percussion can continue via a device or manually. The mist pumped can help clear the tar/fine particles by mechanical flushing action, solvent action as well as by stimulating mucociliary clearance. This method allows a fine mist to clear the lungs, instead of a fluid wash—so the user can be breathing comfortably continuously, without the need for any anesthesia.
6. Device turned off
   This device can be kept on for as long as it takes for the output fluid to come out clear, or in an embodiment, for a predetermined period of time for e.g. 30 minutes.
7. Device sterilization:

The hollow nasopharyngeal tube 14 is sterilized for next use or could be disposable. This cleaning method could be used by the user repeatedly may be once a week/once a month, depending on the severity of deposits, or even few days at a stretch.

In the foregoing specification, specific embodiments of the present invention have been described. However, one of ordinary skill in the art will appreciate that various modifications and changes can be made without departing from the spirit and scope of the present invention as set forth in the various embodiments discussed above and the claims that follow. Accordingly, the specification and figures are to be regarded in an illustrative rather than a restrictive sense, and all such modifications are intended to be included within the scope of present invention. The benefits, advantages, solutions to problems, and any element(s) that may cause any benefit, advantage, or solution to occur or become more pronounced are not to be construed as a critical, required, or essential features or elements as described herein.

We claim:

1. An apparatus comprising:
a hollow nasopharyngeal tube with a longitudinal partition wherein the nasopharyngeal tube is adapted to be placed into a pharynx via a nostril, wherein the hollow nasopharyngeal tube comprises:
a first tubular section for injecting a lung cleansing fluid which forms a fluid mist into a lung, wherein the first tubular section comprises:
a mist generator towards one end for generating the fluid mist to enter the lung;
a second tubular section adapted to provide passage to injected air into the lung along with the fluid mist;
a fluid pump coupled to the first tubular section for pumping the fluid mist into the lung via the first tubular section;
an air pump connected to the second tubular section for pumping air into the lung;
an automated bed comprising a plurality of sides that is configured to pivot around any of the plurality of sides to enable tilting of the bed into a plurality of positions at different angles with respect to a horizontal axis; and
a control unit that signals the automated bed to switch positions in a predetermined sequence on at least one of (a) turning on and off of the fluid pump or the air pump or (b) at least one predetermined interval.

2. The lung cleansing apparatus of claim 1, further comprising:
a cuirass-type negative pressure ventilator adapted to be fixed to at least a part of a thoracic-abdomen and configured to apply a negative pressure in a range of 0 to 50 cm of H₂O and a positive pressure in the range of 0 to 50 cm H2O.

3. The lung cleansing apparatus of claim 1, further comprising:
a heater to preheat at least one of:
the air pumped in via the second tubular section or
lung cleansing fluid pumped in via the first tubular section.

4. The lung cleansing apparatus of claim 1, further comprising:
at least one joint in the automated bed which divides the automated bed into at least two sections out of which at least one section folds in a range of 5 degrees to 90 degrees through the joint in relation to a horizontal plane of the bed.

5. The lung cleansing apparatus of claim 1, further comprising an ultrasonic device coupled to an end of the nasopharyngeal tube.

6. A system comprising:
an automated calibrated bed enabled to change position of an occupant wherein the automated calibrated bed comprises a plurality of sides, and is configured to pivot around any of the plurality of sides to enable tilting of the bed to a plurality of positions at different angles with respect to a horizontal axis;
a pressurized pump device;
a control unit for receiving a signal from the pressurized pump device when the pressurized pump device is turned on, wherein the pressurized pump device is coupled with nasopharyngeal tube, wherein the nasopharyngeal tube comprises:
a first tubular section which is adapted for injecting a fine fluid mist into a lung, wherein the first tubular section comprises a mist generator at one end of the first tubular section for converting a lung cleansing fluid into a fluid mist to enter the lung;
a second tubular section which is used to inject air with predetermined pressure into the lung along with the fluid mist;
a fluid pump coupled to the first tubular section for pumping a lung cleansing fluid into the lung via the first tubular section;
an air pump connected to the second tubular section for pumping air with predetermined pressure into the lung;
changing a position of the automated calibrated bed from a first position around a first axis to a second position around a second axis on an opposite side of the bed on receiving a signal from the control unit.

7. The automated position shifting bed control system of claim 6 further comprising: at least one joint in the automated bed which divides the automated calibrated bed into at least two sections out of which at least one section folds in a range of 5 degrees to 90 degrees through the joint in relation to the horizontal plane of the bed.

8. A method for operating an apparatus, comprising the steps of:
activating, a cuirass-type negative pressure ventilator device that is placed next to at least a part of a thoracic-abdomen with a negative pressure in a range of 0 to 50 cm of H2O and positive pressure range of 0 to 50 cm of H2O;
placing, a hollow nasopharyngeal tube with a longitudinal partition into a pharynx via a nostril, wherein the hollow nasopharyngeal tube comprises:
a first tubular section which is used for injecting a lung cleansing fluid as a fluid mist into a lung, wherein the first tubular section comprises:
a mist generator towards one end for generating fluid mist to enter the lung;
a second tubular section which provides a passage to injected air into the lung along with the fluid mist;
pumping, with a fluid pump connected to the first tubular section the fluid mist into the lungs via the first tubular section;
pumping, with an air pump connected to the second tubular section air with predetermined pressure in the range of 4 cm of H₂O to 6 cm of H₂O into the lungs;
pivoting, an automated bed comprising a plurality of sides, around any of the plurality of sides to enable tilting of the bed to an angle that ranges from 5 to 80 degrees to horizontal axis;
signaling, at a control unit, to switch a position of the automated bed in a predetermined sequence on at least one of (a) turning on and off of the fluid pump or the air pump or (b) on completion of a predetermined interval.

9. The method of claim 8 further comprising preheating, with a heater at least one of:
the air pumped in via the second tubular section; or
the lung cleansing fluid pumped in via the second tubular section.

10. The method of claim 8 further comprising:
changing a position of the automated bed, wherein at least one joint in the automated bed divides the automated bed into at least two sections out of which at least one section folds in a range of 5 degrees to 90 degrees through the joint in relation to a horizontal plane of the bed.
